(19) United States
(12) Patent Application Publication
(10) Pub. No.: US 2025/0082918 A1
(43) Pub. Date: Mar. 13, 2025

(54) ENTERAL FEEDING SYSTEM, METHOD, AND APPARATUS

(71) Applicant: Avent, Inc., Alpharetta, GA (US)

(72) Inventors: Jeffrey S. Nordquist, Lake Barrington, IL (US); Crystal E. Koelper, North Barrington, IL (US); Shawn G. Purnell, Sandy Springs, GA (US); Bernard Daurelle, Marseille (FR)

(21) Appl. No.: 18/953,994

(22) Filed: Nov. 20, 2024

Related U.S. Application Data
(63) Continuation of application No. 15/753,634, filed on Feb. 20, 2018, now abandoned.
(60) Provisional application No. 62/221,845, filed on Sep. 22, 2015.

(51) Int. Cl. A61M 39/10; A61J 15/00; A61M 1/00; A61M 39/12; A61M 39/24

(52) U.S. Cl. CPC A61M 39/105; A61J 15/0003; A61J 15/0073; A61J 15/0092; A61J 15/0096; A61M 1/772; A61M 1/84; A61M 39/1011; A61M 39/12; A61M 39/24; A61M 2039/1033; A61M 2039/1083; A61M 2039/1088; A61M 2205/32; A61M 2210/1053

(57) ABSTRACT

A system for administering nutritional fluids to a patient, including suctioning or aspirating fluids from stomach, performing gastric decompression, and containing gastric reflux. An exemplary system includes a Y-connector, a multiple lumen tube positional in the patient's gastrointestinal track, and an anti-reflux valve for controlling the flow of gastric/reflux material from the system.

### CROSS REFERENCE TO RELATED PATENT APPLICATIONS

This Application is a Continuation of U.S. patent application Ser. No. 15/753,634, filed on Feb. 20, 2018, which is a U.S. National Stage Application filed under 35 U.S.C. § 371 of PCT/US2016/053075, filed on Sep. 22, 2016, which claims the benefit of and priority to U.S. Provisional Application Ser. No. 62/221,845, filed on Sep. 22, 2015, the entireties of which are hereby incorporated by reference herein.

### TECHNICAL FIELD

The present disclosure relates in general to a system, method, and apparatus for administering fluids to body cavities, suctioning or aspirating fluids from body cavities, performing gastric decompression, and containing gastric reflux.

### BACKGROUND

The use of connector ports is commonly required in various medical systems that utilize multiple components to deliver or remove one or more fluids to a patient or other person or animal being treated. One example of such a system is an enteral feeding system in which fluid nutrient formula or the like (e.g., a medication, flushing solution, stomach irrigation, additional nutrient formula) is delivered via a series of tubing segments to a patient. Connectors can also be utilized for continuous or intermittent suctioning, for example, to drain gastric contents or refluxed intestinal secretions, collect stomach aspirate gastric lavage, or decompress gasses from the patient's gastrointestinal tract. In such systems, it may be desirable to remove and collect stomach fluids before, after, or intermittently while fluids are delivered to the patient. The current systems and methods employed in the prior art for medical tubing systems for feeding, suctioning or aspirating fluids, gastric decompression, residual volume measurement, and/or medicating patients may be improved upon as presently disclosed.

### SUMMARY

The present disclosure provides a new and innovative system, method, and apparatus for dual port tube suction and feeding. In an exemplary aspect of the present disclosure, a Y-connector for use in a gastric system includes a delivery arm, a suction arm, a one-way valve, and a convergence arm. The suction arm includes a suction arm stem and an outer end. The suction arm stem has a proximal end and a distal end. The suction arm also includes a tapered hollow stem connector adapted to frictionally fit into a section of tubing. The tapered hollow stem connector has a base and an outlet end, and is axially aligned with the suction arm stem. The base of the hollow stem connector is coupled to the outer end of the suction arm, and the tapered hollow stem connector has an outside diameter that decreases from the base of the hollow stem connector to the outlet end of the hollow stem connector. The one-way valve is interposed between the outlet end of the hollow stem connector and the proximal end of the suction arm stem and the valve is adapted to allow fluid to flow in one direction through the suction arm stem. For example, fluid may flow from the base of the hollow stem connector to the outlet end of the hollow stem connector. The delivery arm has a delivery arm stem, and the delivery arm includes a threaded small-bore connector at an outer end of the delivery arm. The convergence arm has a convergence arm stem. Additionally, the convergence arm includes a connection port adapted to receive a multiple lumen tube. The delivery arm stem is in fluid communication with the convergence arm stem, and the suction arm stem is in fluid communication with the convergence arm stem and the delivery arm stem.

In accordance with another exemplary aspect of the present disclosure, a gastric tubing system includes a Y-connector and a multiple lumen tube. The Y-connector includes a delivery arm, a suction arm, a one-way valve, and a convergence arm. The suction arm includes a suction arm stem and an outer end. The suction arm stem has a proximal end and a distal end. The suction arm also includes a tapered hollow stem connector adapted to frictionally fit into a section of tubing. The tapered hollow stem connector has a base and an outlet end, and is axially aligned with the suction arm stem. The base of the hollow stem connector is coupled to the outer end of the suction arm, and the tapered hollow stem connector has an outside diameter that decreases from the base of the hollow stem connector to the outlet end of the hollow stem connector. The one-way valve is interposed between the outlet end of the hollow stem connector and the proximal end of the suction arm stem, and the valve is adapted to allow fluid to flow in one direction through the suction arm stem. For example, fluid may flow from the base of the hollow stem connector to the outlet end of the hollow stem connector. The delivery arm has a delivery arm stem, and the delivery arm includes a threaded small-bore connector at an outer end of the delivery arm. The convergence arm has a convergence arm stem. Additionally, the convergence arm includes a connection port adapted to receive a multiple lumen tube. The delivery arm stem is in fluid communication with the convergence arm stem, and the suction arm stem is in fluid communication with the convergence arm stem and the delivery arm stem. The multiple lumen tube has an open proximal end and a closed distal end. The open proximal end of the multiple lumen tube is adapted to couple to the connection port on the convergence arm of the Y-connector. Additionally, the multiple lumen tube includes a first lumen and a second lumen that are separated by a wall. The first lumen extends from the proximal end of the multiple lumen tube to the distal end of the multiple lumen tube. The first lumen has an open proximal end and a distal end, the distal end has a plurality of openings. Furthermore, the second lumen has a proximal end and a distal end, the distal end of the second lumen is in fluid communication with the distal end of the first lumen and has a fluid communication opening on the wall between the first lumen and second lumen. The communication opening is adapted to allow air to enter the distal end of the first lumen. The proximal end of the second lumen extends beyond the proximal end of the first lumen, and the proximal end of the second lumen is in fluid communication with the ambient air.

In accordance with another exemplary aspect of the present disclosure, which may be used in combination with any one or more of the preceding aspects, an enteral feeding and suction system includes a fluid source, a suction assembly, a Y-connector, a multiple lumen tube, an administration tubing segment and a suction tubing segment. The suction assembly includes a vacuum and a suction canister. Additionally, the vacuum and the suction canister are connected by a section of vacuum tubing. The Y-connector includes a delivery arm, a suction arm, a one-way valve, and a convergence arm. The suction arm includes a suction arm stem and an outer end. The suction arm stem has a proximal end and a distal end. The suction arm also includes a tapered hollow stem connector adapted to frictionally fit into a section of tubing. The tapered hollow stem connector has a base and an outlet end, and is axially aligned with the suction arm stem. The base of the hollow stem connector is coupled to the outer end of the suction arm, and the tapered hollow stem connector has an outside diameter that decreases from the base of the hollow stem connector to the outlet end of the hollow stem connector. The one-way valve is interposed between the outlet end of the hollow stem connector and the proximal end of the suction arm stem and the valve is adapted to allow fluid to flow in one direction through the suction arm stem. For example, fluid may flow from the base of the hollow stem connector to the outlet end of the hollow stem connector. The delivery arm has a delivery arm stem, and the delivery arm includes a threaded small-bore connector at an outer end of the delivery arm. The convergence arm has a convergence arm stem. Additionally, the convergence arm includes a connection port adapted to receive a multiple lumen tube. The delivery arm stem is in fluid communication with the convergence arm stem, and the suction arm stem is in fluid communication with the convergence arm stem and the delivery arm stem. The multiple lumen tube has a proximal end and a distal end. The multiple lumen tube includes a first lumen and a second lumen. The first lumen has a proximal end and a distal end. The distal end has at least one opening. The proximal end of the first lumen is adapted to attach to the connection port on the convergence arm of the Y-connector. The second lumen has a proximal end and a distal end. The distal end of the second lumen is in fluid communication with the distal end of the first lumen and has an opening on the wall between the first lumen and the second lumen adapted to allow air to enter the distal end of the first lumen. The proximal end of the second lumen extends beyond the proximal end of the first lumen, and the proximal end of the second lumen is in fluid communication with the ambient air and has an enlarged diameter. The administration tubing segment has a first end connected to the fluid source and a second end adapted to connect to the primary arm of the Y-connector. The suction tubing segment has a first end and a second end. The first end of the suction tubing segment is connected to the suction canister, and the second end of the suction tubing segment is adapted to connect to the tapered hollow stem connector on the suction arm.

In accordance with another exemplary aspect of the present disclosure, which may be used in combination with any one or more of the preceding aspects, an enteral feeding and gastric pressure relief system includes a gastric material collection reservoir, a fluid source, a Y-connector, a multiple lumen tube, an administration tubing segment, and a relief tubing segment. The gastric material collection reservoir is adapted to collect reflux fluids from the patient's stomach. The Y-connector includes a delivery arm, a suction arm, a one-way valve, and a convergence arm. The suction arm includes a suction arm stem and an outer end. The suction arm stem has a proximal end and a distal end. The suction arm also includes a tapered hollow stem connector adapted to frictionally fit into a section of tubing. The tapered hollow stem connector has a base and an outlet end, and is axially aligned with the suction arm stem. The base of the hollow stem connector is coupled to the outer end of the suction arm, and the tapered hollow stem connector has an outside diameter that decreases from the base of the hollow stem connector to the outlet end of the hollow stem connector. The one-way valve is interposed between the outlet end of the hollow stem connector and the proximal end of the suction arm stem and the valve is adapted to allow fluid to flow in one direction through the suction arm stem. For example, fluid may flow from the base of the hollow stem connector to the outlet end of the hollow stem connector. The delivery arm has a delivery arm stem, and the delivery arm includes a threaded small-bore connector at an outer end of the delivery arm. The convergence arm has a convergence arm stem. Additionally, the convergence arm includes a connection port adapted to receive a multiple lumen tube. The delivery arm stem is in fluid communication with the convergence arm stem, and the suction arm stem is in fluid communication with the convergence arm stem and the delivery arm stem. The multiple lumen tube includes a first lumen and a second lumen. The first lumen has a proximal end and a distal end. The distal end has at least one opening. The proximal end of the first lumen is adapted to attach to the connection port on the convergence arm of the Y-connector. The second lumen has a proximal end and a distal end. The distal end of the second lumen is in fluid communication with the distal end of the first lumen and has an opening on the wall between the first lumen and the second lumen adapted to allow air to enter the distal end of the first lumen. The proximal end of the second lumen extends beyond the proximal end of the first lumen, and the proximal end of the second lumen is in fluid communication with the ambient air and has an enlarged diameter. The administration tubing segment has a first end connected to the fluid source and a second end adapted to connect to the delivery arm of the Y-connector. The relief tubing segment has a first end and a second end. The first end of the relief tubing segment is connected to the gastric material collection reservoir. Additionally, the second end of the relief tubing segment is connected to the open proximal end of the second lumen of the multiple lumen tube.

Additional features and advantages of the disclosed system, method, and apparatus are described in, and will be apparent from, the following Detailed Description and the Figures.

### BRIEF DESCRIPTION OF THE FIGURES

FIG. 1A is a perspective view of a nasogastric/nasointestinal enteral suction system implementing a dual port tube for suction and feeding, according to an example embodiment of the present disclosure.

FIG. 1B is a perspective view of a nasogastric/nasointestinal enteral feeding system implementing a dual port tube for suction and feeding, according to an example embodiment of the present disclosure.

FIG. 1C is a perspective view of a nasogastric/nasointestinal enteral feeding and gastric pressure relief system implementing a dual port tube for suction and feeding, according to an example embodiment of the present disclosure.

FIG. 2 is a perspective view of an enteral feeding and gastric pressure relief device, according to an example embodiment of the present disclosure.

FIG. 3A is a side view of a gastric tubing apparatus with an anti-reflux valve removed, according to an example embodiment of the present disclosure.

FIG. 3B is a side view of a gastric tubing apparatus, according to an example embodiment of the present disclosure.

FIG. 3C is an enlarged front view of a multiple lumen tube, according to an example embodiment of the present disclosure.

FIG. 3D is an enlarged cross-sectional view of a multiple lumen tube, according to an example embodiment of the present disclosure.

FIG. 4A is a cross-sectional view of a multiple lumen tube of the gastric tubing system, according to an example embodiment of the present disclosure.

FIG. 4B is a cross-sectional view of a multiple lumen tube of the gastric tubing system, according to an example embodiment of the present disclosure.

FIG. 4C is a cross-sectional view of a multiple lumen tube of the gastric tubing system, according to an example embodiment of the present disclosure.

FIG. 4D is a cross-sectional view of a multiple lumen tube of the gastric tubing system, according to an example embodiment of the present disclosure.

FIG. 5A is a side view of a Y-connector, according to an example embodiment of the present disclosure.

FIG. 5B is a side view of a Y-connector, according to an example embodiment of the present disclosure.

FIG. 5C is an enlarged top view of a securable Y-connector cap.

FIG. 5D is a cross-sectional view of a gastric tubing apparatus with a positioning stylet, according to an example embodiment of the present disclosure.

FIG. 5E is a side view of a Y-connector with a stopcock valve, according to an to an example embodiment of the present disclosure.

FIG. 5F is a side view of a Y-connector with a stopcock valve, according to an example embodiment of the present disclosure.

FIG. 6A is a side view of a Y-connector inner core, according to an example embodiment of the present disclosure.

FIG. 6B is a cross-sectional view of a Y-connector with a material over-layer shown in phantom, according to an example embodiment of the present disclosure.

FIG. 7 is a side view of a Y-connector material over-layer, according to an example embodiment of the present disclosure.

FIG. 8A is a side view of an anti-reflux valve attached to a connector, according to an example embodiment of the present disclosure.

FIG. 8B is an exploded side view of an anti-reflux valve and a connector, according to an example embodiment of the present disclosure.

FIG. 9A is a flowchart of an example suction and feeding process using a nasogastric enteral suction system and feeding system.

FIG. 9B is a flowchart of example process using an enteral feeding and gastric pressure relief device.

### DETAILED DESCRIPTION OF EXAMPLE EMBODIMENTS

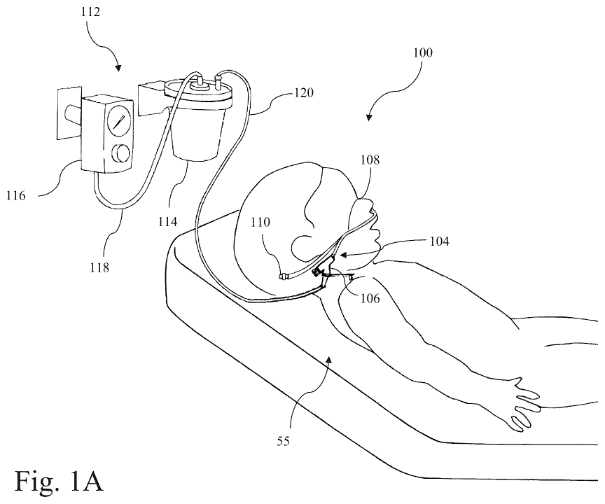

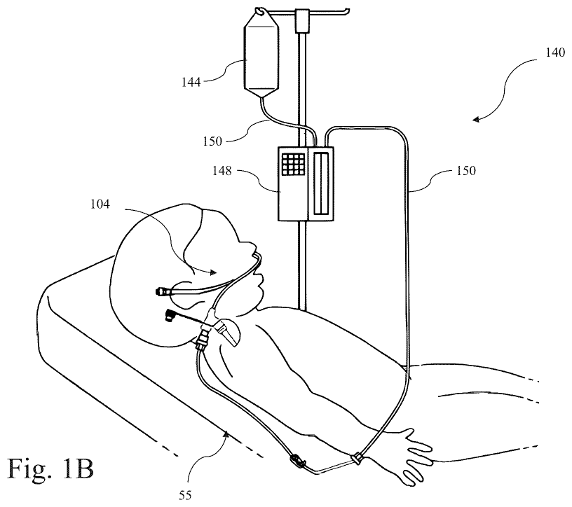

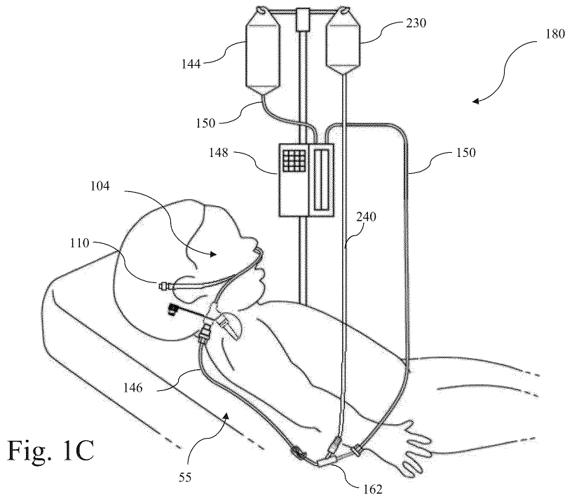

Referring to the Figures, embodiments of an enteral feeding and gastric pressure relief/suction device are disclosed. As seen in an exemplary enteral suction system 100 illustrated in FIG. 1A, a gastric tubing apparatus 104 is used in conjunction with a suction assembly 112. The suction assembly 112 may comprise a suction canister 114, a vacuum 116, and a section of vacuum tubing 118. In the illustrated embodiment, the vacuum 116 may be a wall-mounted vacuum or a portable vacuum system. A typical wall-mounted vacuum will produce between 550 and 760 mm Hg of suction. The vacuum may create a suction pressure in the low pressure range (e.g., 0 to 80 mm Hg), medium pressure range (e.g., 80 to 120 mm Hg), or high pressure range (e.g., greater than 120 mm Hg). However, it is typically recommended that a patient 55 is exposed to the least amount of vacuum pressure possible for the clinical need, and guidelines state suction pressures typically between 30-40 mm Hg for nasogastric suctioning (reference Brunner and Suddarth's Textbook of Medical-Surgical Nursing: 12th edition (2009), page 1022). Additionally, intermittent nasogastric suctioning is recommended rather than continuous mode operation of the vacuum system. The wall-mounted vacuum may be controlled with a digital controller or a regulator dial so that the proper suction pressure may be applied through the vacuum tubing 118. The flow rate of gastric material or gastric fluid from the patient's stomach to the suction canister 114 may be accomplished by using the vacuum 116 in combination with the use of suction tubing 120. The term “fluid” as used herein refers to and includes both gaseous and liquid physical states, which may also include solid matter. The suction tubing 120 may be attached to the gastric tubing apparatus 104 during a suction procedure on the patient 55. Once connected, the vacuum 116 may be activated and suction through the suction tube set 120 may be applied at the appropriate suction pressure (e.g., low, medium, or high). Once suction has started, gastric material or gastric fluid enters the gastric tubing apparatus 104 and passes through the suction tubing 120 where it is then separated and collected by the suction canister 114. The gastric tubing apparatus 104 includes a multiple lumen tube 108, a y-connector 106, and may also include an anti-reflux valve 110. The multiple lumen tube 108 may be made from polyurethane, silicone, PVC, or the like. Additionally, the multiple lumen tube 108 may include a vent tube 410 (described in further detail below). Venting helps protect against damage to the gastric mucosa, mucosal tissue tears, bleeding, and increased risk of infections while suction/drainage is occurring as atmospheric air is drawn into the second lumen to equalize the suction vacuum pressure in the stomach once the contents have been emptied. In an example embodiment, the anti-reflux valve 110 is oriented upward to reduce the chance of clogging or wetting of the filter material in the anti-reflux valve 110. This orientation may be achieved by using a clothing clip or other attachment means connected to the multiple lumen tube 108 such that the anti-reflux valve 110 maintains a proper position during treatment. For example, the clothing clip may attach to a pillow or a shirt collar on the patient 55. Other enteral suction arrangements and configurations can be used in connection with the embodiments of the present disclosure illustrated and discussed herein, as will be appreciated by those skilled in the art.

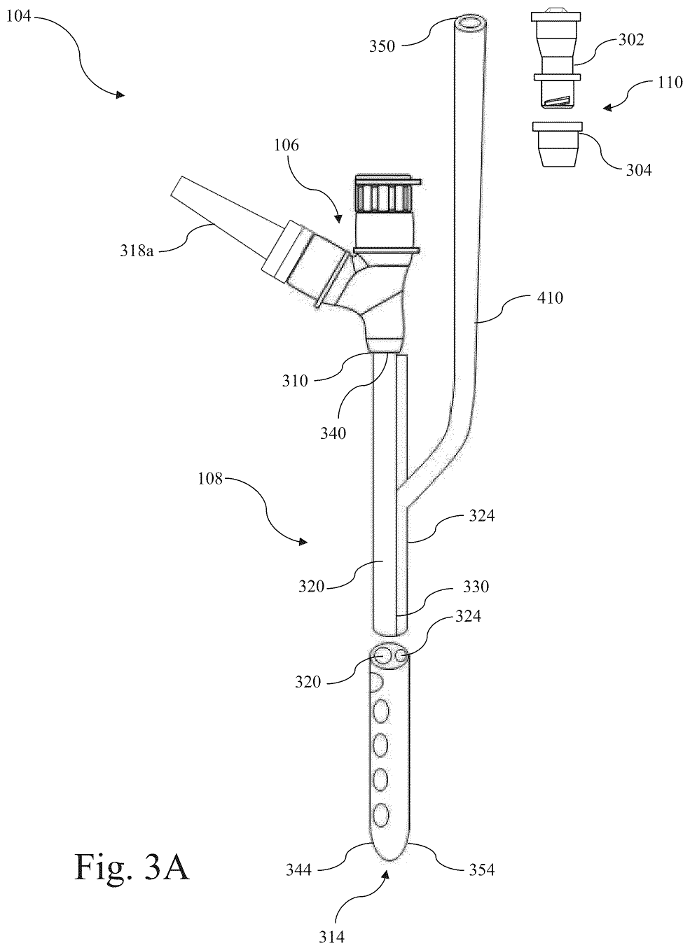

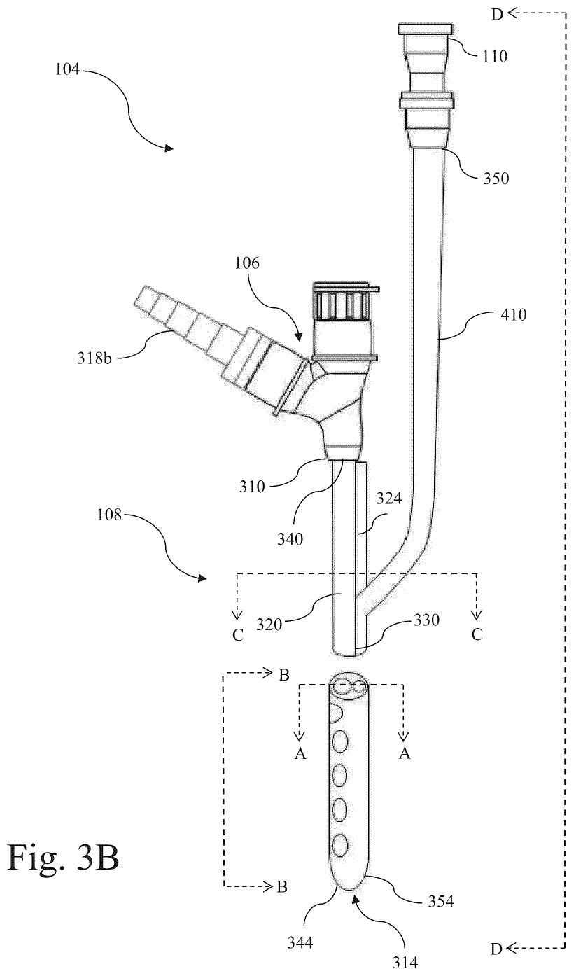

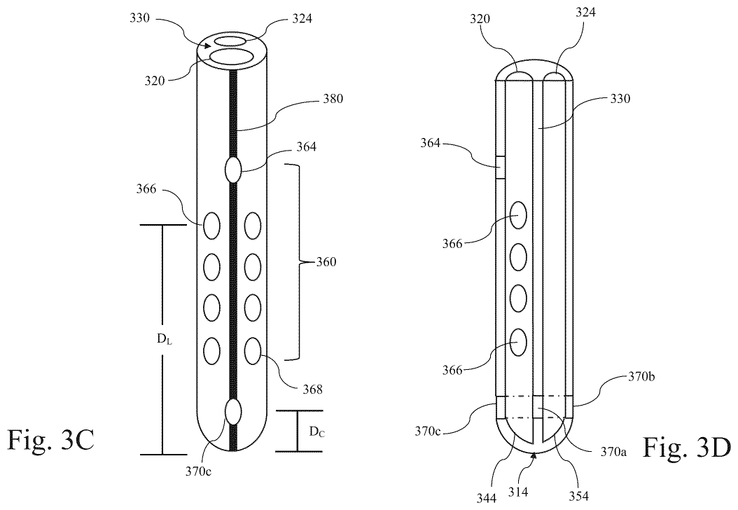

FIG. 1B illustrates an example embodiment of an enteral feeding system 140. Nutrient formula is provided from a feeding container 144 (e.g., a suspended feeding bag) and is pumped using pump 148 via administration tubing 150. In an example embodiment, the pump 148 may be a peristaltic pump, however, nutrient formula delivery may also be accomplished without a pump (e.g., using gravity feeding). The administration tubing 150 may be attached to the gastric tubing apparatus 104 to initiate a feeding procedure on the patient 55. A detail of the multiple lumen suction and feeding connector 104 from FIGS. 1A and 1B is described in more detail in FIG. 3.

FIG. 1C illustrates an example embodiment of an enteral feeding and gastric pressure relief system 180. Nutrient formula is provided from a feeding container 144 (e.g., bag) and is pumped using pump 148 via administration tubing 150. In an example embodiment, the pump 148 may be a peristaltic pump, however, nutrient formula delivery may also be accomplished without a pump (e.g., using gravity feeding). Gastric pressure relief (e.g., gastric decompression) may be provided using a collection reservoir 230 and relief tubing 240 connected to a multi-way connector 162. The relief tubing 240 connects the collection reservoir 230 to a multi-way connector 162. Fluid delivery tubing 146 and administration tubing 150 are also connected to the multi-way connector 162. The fluid delivery tubing 146 may be attached to the gastric tubing apparatus 104 to initiate a feeding procedure on the patient 55. An example embodiment, of the multiple lumen suction and feeding connector 104 from FIGS. 1A and 1B is described in more detail in FIG. 3. In an example embodiment, the anti-reflux valve 110 may be capped or closed to ensure that reflux from the patient passes through the gastric tubing apparatus 104, to the fluid delivery tubing 146 and through the multi-way connector 162 into the relief tubing 240 and into the collection reservoir 230.

Additionally, a one-way check valve or other backflow prevention means can be integrated into the enteral feeding and gastric pressure relief system 180 to prevent retrograde flow back into administration tubing 150 (and thus into feeding container 144). The one-way check valve can be an internal valve in one arm of the multi-way connector 162, or it can be interposed between the multi-way connector 162 and administration tubing 150. The one-way check valve permits flow only in one direction (i.e., toward relief tubing 240 and/or fluid delivery tubing).

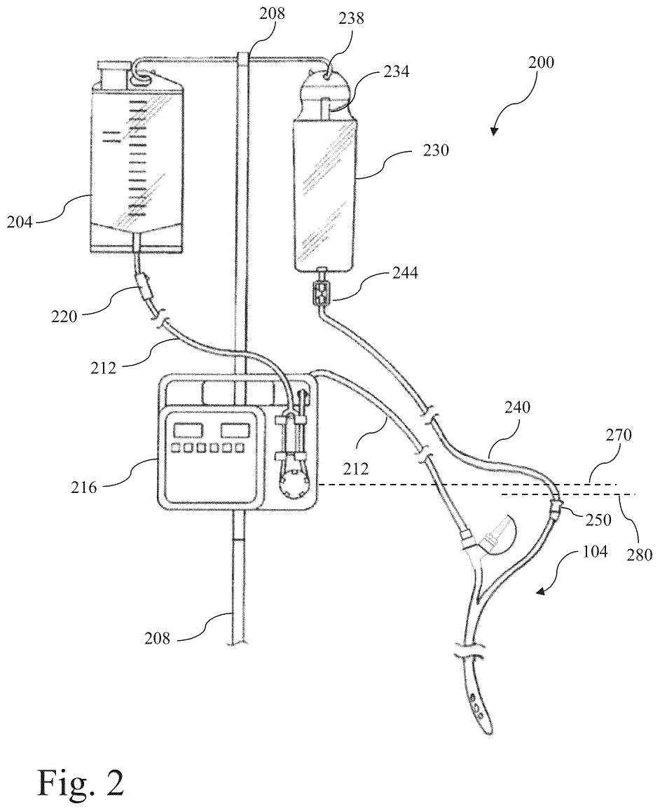

FIG. 2 is a perspective view of an enteral feeding and gastric pressure relief device 200. An enteral feeding container 204 containing a selected nutrient formula is suspended from a support stand 208. Other enteral feeding system arrangements and configurations can be used in connection with the embodiments of the present disclosure illustrated and discussed herein, as will be appreciated by those skilled in the art. The flow rate for delivery of nutrient formula from the enteral feeding container 204 through administration tubing 212 may be accomplished through use of an enteral feeding pump 216 in combination with the use of an administration tube set clamping mechanism 220. Nutrient formula flow rate may also be achieved through gravity feed controlled through the combination of a drip chamber and a tubing clamp (e.g., a roller or other adjustable clamp).As explained in greater detail below, gastric pressure relief is achieved through the appropriate connection between the vent lumen of gastric tubing apparatus 104, the relief tubing 240, and the collection reservoir 230. The reflux material collection reservoir 230 is configured to receive materials (i.e., gas and/or liquid) refluxed from a patient's stomach during gastric pressure relief. In FIG. 2, the collection reservoir 230 is vented to the ambient atmosphere through a gas vent 234 and is suspended from the support stand 208 by a hanger tab 238. A segment of relief tubing 240 connects the collection reservoir 230 to a gastric tubing apparatus 104; a relief tubing clamping mechanism 244 can be used to selectively control the flow of gases and liquids between the gastric tubing apparatus 104 and the collection reservoir 230. Administration tubing 212 is also connected to the gastric tubing apparatus 104. In an example embodiment, the relief tubing 240 has a connector 250 adapted to connect to an air-vent lumen from the gastric tubing apparatus 104, which will be described further below. For example, if an anti-reflux valve 110 is connected to the air-vent lumen on the gastric tubing apparatus 104, then the anti-reflux valve 110 may be removed and the connector 250 may be attached to the air-vent lumen. In an example embodiment, the connector 250 may be a female mating threaded small-bore connector (e.g., ISO 80369-3).

The relief apparatus as illustrated in FIG. 2 comprises a collection reservoir 230, which is vented to ambient atmospheric pressure. To avoid introducing air into the enteral feeding tube through the relief tubing segment and administration tubing segment, the gastric pressure relief apparatus 200 is configured to provide a small column of liquid (e.g., nutrient formula or other liquid) in the relief tubing segment 240 or in the air-vent lumen. This is accomplished by properly positioning the connection between the air-vent lumen of the gastric tubing apparatus 104 and the relief tubing 240 at or slightly below the level of a patient's stomach. For reference and illustrative purposes, the patient's stomach level is indicated in FIG. 2 by line 270. For example, if the connection between the air-vent lumen of the gastric tubing apparatus 104 and the relief tubing 240 is at or below that patient stomach level 270 (or, e.g., the patient's mid-axillary line), a small column of liquid (e.g., nutrient formula or other liquid) enters and remains suspended in the relief tubing 240 with the meniscus of the column generally at line 280 in FIG. 2 (in close proximity to, or at the same level as the stomach level 270, typically based on the physical condition of the patient). This liquid column prevents air from being drawn into the administration tubing.

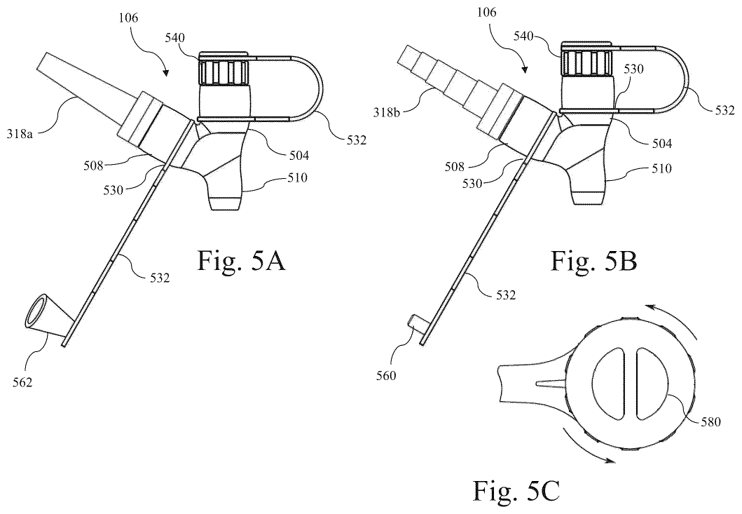

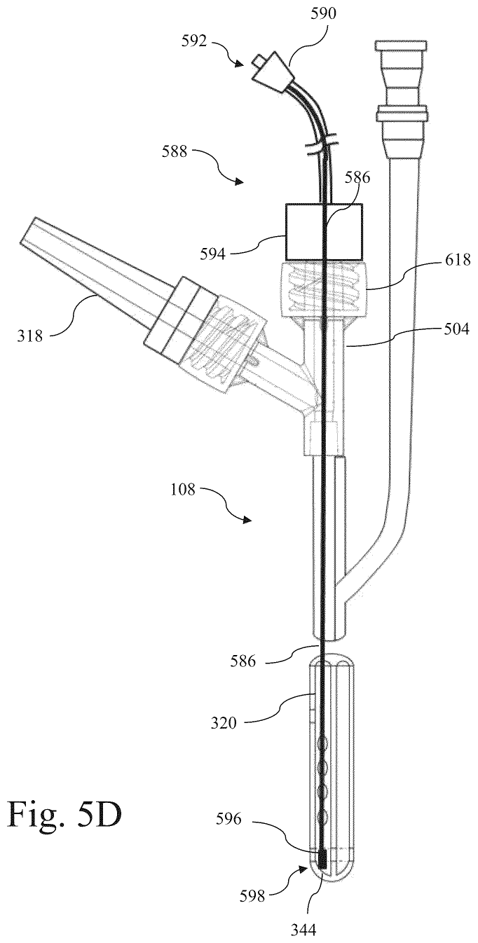

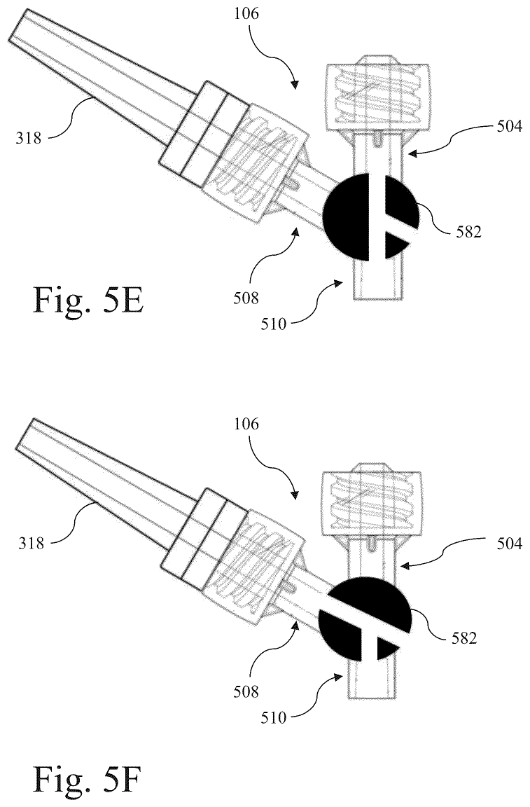

FIGS. 3A and 3B illustrate side views of a gastric tubing apparatus 104. In FIG. 3A, the gastric tubing apparatus 104 is shown with the anti-reflux valve 110 removed. In an example embodiment, the anti-reflux valve 110 may include an anti-reflux filter housing 302, which is adapted to fit into an associated connector 304. For example, connector 304 may be a male enteral only connector (e.g., ISO 80369-3). The gastric tubing apparatus 104 includes a Y-connector 106 coupled to a multiple lumen tube 108 with an open proximal end 310 and a closed distal end 314. The Y-connector 106 also includes a suction connector 318. In an example embodiment, the suction connector 318 may be a constant tapered hollow stem connector 318a, or in another example embodiment, the suction connector 318 may be a stepwise tapered hollow stem connector 318b. The suction connector 318 is tapered, which allows it to frictionally press-fit into the suction tubing 120. In an example embodiment, the suction connector 318 and the remaining structure of the Y-connector may be formed as single, unitary plastic structure. In other embodiments, the suction connector 318 can be affixed to the remaining structure of the Y-connector 106 using adhesive, solvent bonding, radio-frequency (RF) welding and/or any other suitable means of affixing the suction connector 318 to the Y-connector 106. For example, as illustrated in FIGS. 5E and 5F, the suction connector 318 may be threaded onto the remaining structure of the Y-connector 106. The suction connector 318 may be manufactured of a rigid material such as a suitable copolyester, polyamide, polyethylene, polypropylene, polyacrylonitrile, or the like. An amorphous copolyester, polyamide, polyethylene, polypropylene, polyacrylonitrile, or similar material product typically has suitable appearance, clarity and mold release properties (usable with injection molding, for example). It also provides appropriate rigidity, toughness/durability, hydrolytic stability, heat resistance, and chemical resistance and has been formulated for medical devices.

The multiple lumen tube 108 includes a first lumen 320 and a second lumen 324 separated by a wall 330. The first lumen 320 has a proximal end 340 that is open and a distal end 344 that is closed. The proximal end 340 of the first lumen 320 is coupled to Y-connector 106. The second lumen 324 has a proximal end 350 and a distal end 354. The distal end 354 of the second lumen 324 is in fluid communication with the distal end 354 of the first lumen 320. The proximal end 350 of the second lumen 324 extends beyond the proximal end 340 of the first lumen 320. Additionally, the proximal end 350 of the second lumen 324 is in fluid communication with ambient air and may have an enlarged diameter. The anti-reflux valve 110 is coupled to the proximal end 350 of the second lumen 324.

FIG. 3C and FIG. 3D show an enlarged view of the multiple lumen tube 108. FIG. 3C illustrates an enlarged sectional view along the line B-B in FIG. 3B. FIG. 3D illustrates an enlarged cross-sectional view along line A-A in FIG. 3B. The multiple lumen tube 108 is a flexible hollow tube constructed from a suitable material adapted to maintain flexibility and provide enough structural support to prevent closure or blowout of one of the multiple lumens (320, 324) during suction and/or feeding. Unlike rigid tubes or tubes made from materials that stiffen throughout application, an example embodiment of the multiple lumen tube 108 is produced from a material that maintains flexibility throughout use and advantageously reduces injury to the patient during insertion and removal of the tube. For example, the use of more rigid tubes or tubes that lose flexibility and harden may cause serious problems including necrosis at the nare, damage to the vocal chords, or cause the patient other irritation and discomfort or injury. In an example embodiment, the multiple lumen tube 108 may be made from polyurethane, silicone, or the like, which advantageously provide sufficient flexibility, durability, chemical resistant properties, and ease of molding. In another example embodiment, the multiple lumen tube 108 may be made from polyurethane because PVC may stiffen over time when exposed to the acid environment of the stomach. Instead, polyurethane is “softer” with increasing temperature (e.g., as the temperature increases, the polyurethane becomes more elastic).

As shown in FIG. 3C, the multiple lumen tube 108 may also include a radiopaque stripe 380 that extends the length of the tube. In an example embodiment, the radiopaque stripe 380 may be on the outer wall of the first lumen 320. For example, the radiopaque stripe 380 may be a color stripe containing a radiopaque material such as barium sulfate. The radiopaque stripe 380 advantageously enhances the radiopacity of the multiple lumen tube 108 and allows the tube to be visible on x-ray film, which helps a clinician properly confirm location of the tube. In an example embodiment, the radiopaque stripe 380 may cover 25 percent to 50 percent of the outer wall of the first lumen 320. In another example embodiment, the radiopaque stripe 380 may cover 40 percent of the outer wall of the first lumen 320.

The distal end 344 of the first lumen 320 has a plurality of openings 360. The plurality of openings 360 are arranged to provide a gastric material inlet to the first lumen 320 during suction, and provide a nutrient formula outlet of the first lumen 320 during feeding. In an example embodiment, the plurality of openings 360 may include a location hole 364, a first pattern of holes 366, and a second pattern of holes 368. The location hole 364 is placed furthest from the closed distal end 314 of the multiple lumen tube 108 at a predetermined distance (D_{L}) and cuts through the first lumen 320 of the multiple lumen tube 108 to interrupt the radiopaque stripe 380. The interruption of the radiopaque stripe by the location hole 364 advantageously allows the position of the multiple lumen tube 108 to be located using an x-ray to confirm location of this opening. Specifically, the radiopaque stripe 380, the location hole 364, and the distance D_{L} help a clinician determine the position of the closed distal end 314 of the multiple lumen tube 108 because the location hole 364 causes an interruption in the radiopaque stripe 380 and also an interruption in the x-ray visibility. The first pattern of holes 366 and the second pattern of holes 368 may be positioned along each side of the first lumen 320 tube. Additionally, the first pattern of holes 366 may be axially aligned with the second pattern of holes 368. The first pattern of holes 366 and the second pattern of holes 368 are positioned on opposite sides of the first lumen tube to reduce the chance of the plurality of openings 360 from sticking to the stomach wall during the suctioning process, which advantageously reduces the chances of causing a tissue tear, bleeding, gastric mucosal damage, and increased risk of infection. The holes may be oval (as depicted in FIG. 3C), circular, or other geometric shapes which are easy to fabricate with the ability to have smooth transitions to the outer tube surface.

In an example embodiment, there may be a fluid communication opening 370 in the wall between the first lumen and the second lumen adapted to allow air to enter the distal end 344 of the first lumen 320 (shown as 370a). The fluid communication opening 370 is placed closest to the closed distal end 314 of the multiple lumen tube 108 at a predetermined distance (D_{C}). Placement of the fluid communication opening 370 closest to the closed distal end 314 of the multiple lumen tube 108 advantageously ensures that ventilation and pressure equalization is provided in the multiple lumen tube 108 at all insertion depths. In an example embodiment, the fluid communication opening 370 may also extend through the outer wall of the second lumen 324 (shown as 370b). Furthermore, in another example embodiment, the fluid communication opening 370 may extend through the outer wall of the second lumen 324, the wall 330 between the first lumen and the second lumen, and the outer wall of the first lumen 320 (shown as 370c). The fluid communication opening 370c may be placed closest to the closed distal end 314 of the multiple lumen tube 108 and may cut through the first lumen 320 of the multiple lumen tube 108 to interrupt the radiopaque stripe 380. The interruption of the radiopaque stripe by the fluid communication opening 370c advantageously allows the position of the most distal fluid communication opening 370c to be located using an x-ray. Location hole 364 and fluid communication opening 370c interrupting the radiopaque stripe may be used independently (e.g., only one hole/opening interrupting the stripe) or together in certain design configurations.

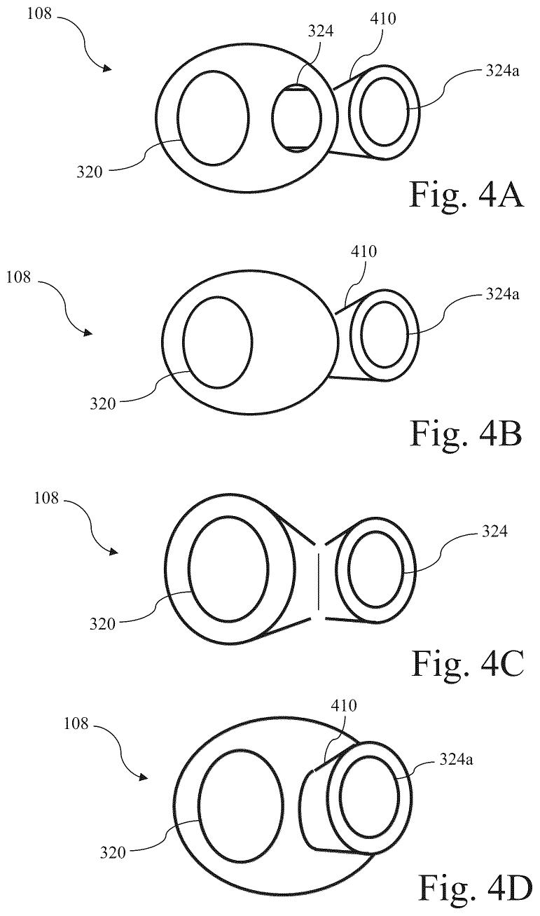

FIGS. 4A through 4D show cross-sectional views of the multiple lumen tube 108 of the gastric tubing system. The multiple lumen tube 108 may have each of the lumens fully extruded through the proximal end 310 of the multiple lumen tube 108, as shown in FIG. 4A. The multiple lumen tube 108 may also include a vent tube 410 that is inserted into second lumen 324 through the outer wall of the multiple lumen tube 108. Venting helps protect against damage to the gastric mucosa, mucosal tissue tears, bleeding, and increased risk of infections while suction/drainage is occurring as atmospheric air is drawn into the second lumen to equalize the suction vacuum pressure in the stomach once the contents have been emptied. In an example embodiment, the vent tube 410 is adapted to block the second lumen 324 passage to the proximal end 310 of the multiple lumen tube 108 and reroute the second lumen 324a passage from the distal end 314 of the multiple lumen tube 108 through the vent tube 410. The vent tube 410 extends out from the side of the second lumen 324 tube of the multiple lumen tube 108 and has a proximal end 350 that extends beyond the proximal end 340 of the first lumen 320, as illustrated in FIG. 3A.

Additionally, as shown in FIG. 4B, the multiple lumen tube 108 may consist of a first lumen 320 that has been extruded through the proximal end 310 of the multiple lumen tube 108 and a second lumen 324 extruded to a predetermined height from the proximal end 310 of the multiple lumen tube 108. A vent tube 410 may then couple to the multiple lumen tube 108 at the location where the second lumen 324 extrusion ends to extend the passage of the second lumen 324a from the multiple lumen tube 108 through the vent tube 410.

As shown in FIG. 4C, the first lumen 320 and the second lumen 324 of the multiple lumen tube 108 may include two separate lumen tubes that are bonded together. For example, the lumen tubes may be joined together with a biocompatible adhesive, bonded by sonic welding, melted together, or bonded using any other suitable means.

As shown in FIG. 4D, the multiple lumen tube 108 may include a first lumen 320 and a second lumen 324a that have been extruded through the proximal end 310 of the multiple lumen tube 108. Additionally, the second lumen 324a may extend through a vent tube 410 that is coupled to the proximal end 310 of the multiple lumen tube 108. For example, the vent tube 410 may be coupled to the multiple lumen tube 108 so that the second lumen 324a extends beyond the proximal end 310 of the multiple lumen tube 108.

As shown in FIGS. 5A and 5B, the Y-connector 106 has a delivery arm 504 and a suction arm 508, both of which are connected to a convergence arm 510. It should be appreciated that each arm may also be referred to as a “port”. For example, the convergence arm 510 may be referred to as a convergence port or a bidirectional port. The convergence arm 510 has a generally cylindrical stem and is the lower part of the “Y” shape of the Y-connector 106.

In an example embodiment, the delivery arm 504 and convergence arm 510 are generally coaxial, providing a generally linear flow path (of varying diameters in some embodiments) for fluids flowing between the delivery arm 504 and the convergence arm 510. The suction arm 508 is of similar construction to the delivery arm 504, but is oriented at an angle of approximately 60° relative to the delivery arm 504 and convergence arm 510 in the non-limiting exemplary embodiments of the Figures. Other angular orientations between the delivery arm 504 and suction arm 508 may also be implemented. Generally, the likelihood of clogging or other improper flow problems may increase as the angle of the suction arm 508 gets closer to 90°. In some uses, performance may be reduced or otherwise affected with changes in the angular orientation.

The Y-connector 106 may also include a ring connector 530 on each arm. The ring connector 530 or other suitable connector allows a plug or cap to be tethered or otherwise attached to the delivery arm 504. In an example embodiment, the ring connector 530 may include a tether 532 to attach a cap or a plug to each arm. For example, the delivery arm 504 may include a ring connector 530 with tether 532 that is attached to a delivery arm connector cap 540. Additionally, the suction arm 508 may include a ring connector 530 with tether 532 that is attached to a suction connector plug 560 or suction connector cap 562. In an example embodiment, the suction connector cap 562 may also include a plug such that the connector cap 562 seals both the inside and the outside of the suction connector 318.

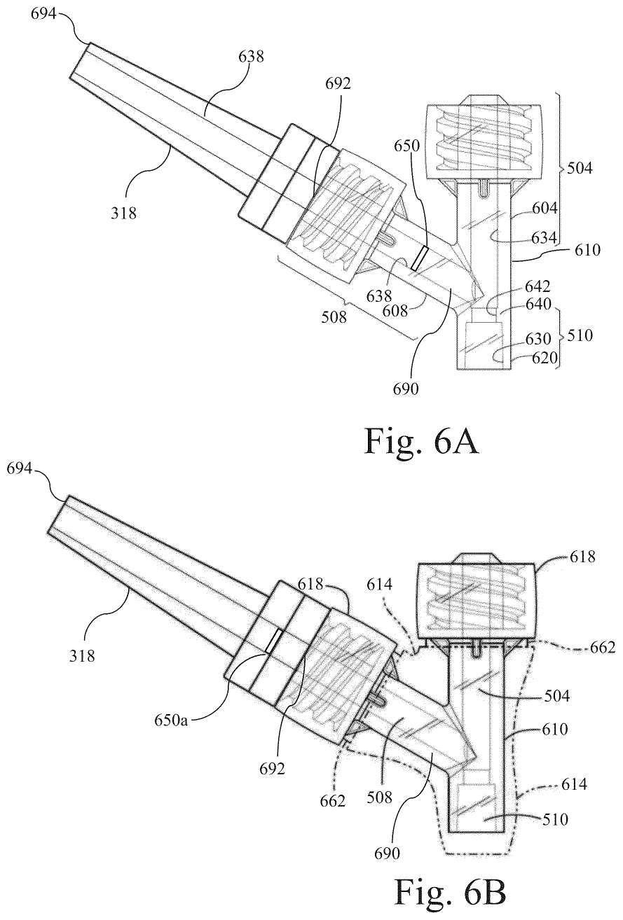

The delivery arm 504 small-bore connector cap 540 is threaded and adapted to engage the mating threaded small-bore connector 618 in FIG. 6B. The configuration of the small-bore connector 618 and the small-bore connector cap 540 conform to applicable national and/or international standards (e.g., ISO 80369-3 relative to connectors for enteral applications) for such connectors and the like. Additionally, the delivery arm 504 may be utilized to check properties of the stomach contents (e.g. pH, gastric fluid sampling, etc.) using an enteral syringe. In an example embodiment, the small-bore connector 618 may be configured to be accessible by a syringe or other fluid withdrawal and pumping (e.g., “manmade force-applying”) means capable of withdrawing gastric material or reflux material contained in the Y-connector 106 (for example, using an ISO 80369-3 connection configuration). For example, a clinician may fit the end of the small-bore connector 618 with an enteral syringe to perform a pH check of the stomach contents.

The interior of the delivery arm 504 small-bore connector cap 540 may utilize whatever structure is effective to seal an inlet/outlet to prevent ingress of unwanted material into and egress of internal materials from a system incorporating one or more Y-connectors according to the embodiments. The external configuration of the cap enhances ease of use for a nurse, clinician, etc. The cylindrical sidewalls on the periphery of the cap may have gripping means (e.g., knurling, grips, ridges or the like) to provide better gripping of the cap, especially if it is wet or has adhered material that makes disengagement from and/or securement to the Y-connector difficult.

Additionally, the interior of the suction arm suction connector cap may utilize whatever structure is effective to seal the inlet/outlet to prevent ingress of unwanted material into an egress of internal materials from a system. In an example embodiment, a suction connector plug may be used that is adapted to press-fit inside the suction arm channel 638 (as illustrated in FIG. 6A). For example, the suction connector plug 560 may be made of rubber or other suitable compressible material such that it can create an airtight seal at the end of the suction connector 318.

As shown in FIG. 5C, the top of the cap may have a double-D configuration 580. This type of multi-indentation cap configuration, which the top of the cap has multiple indentations, recesses, cavities or the like, allows a nurse, clinician, etc. to use a tool (e.g., a cleat or pick) to assist in dislodging a stuck cap (e.g., if a cap was encrusted with material making cap disengagement difficult). Moreover, the multi-indentation cap configuration is advantageous from a fabrication perspective, for example by reducing manufacturing and mold processing challenges.

The delivery arm 504 (e.g., feeding arm) may be aligned with the convergence arm 510 to advantageously allow a positioning stylet 586 of a tube assembly guidance system 588 to be used with the gastric tubing apparatus 104. For example, FIG. 5D illustrates a cross-sectional view of a gastric tubing apparatus with a positioning stylet along line D-D in FIG. 3B. In an example embodiment, the positioning stylet 586 of a tube assembly guidance system 588, such as the CORTRAK® enteral access system, may be used with the delivery arm 504 of the gastric tubing apparatus 104 to assist in positioning the closed distal end 314 of the multiple lumen tube 108 within the patient 55. In an example embodiment, the positioning stylet 586 has an electrical connector 590 included at a first end 592, a union device 594 adapted to couple to the small-bore connector 618 of the delivery arm 504, and a coil 596 included at a second end 598 of the positioning stylet 586. The first end of the positioning stylet 586 may be adapted to couple to the small-bore connector 618 of the delivery arm 504. Additionally, the second end 598 of the positioning stylet 586 may be adapted to move through the first lumen 320 of the multiple lumen tube 108, and the second end 598 may be located within the first lumen 320 and positioned at the distal end 344 of the first lumen 320. For example, the positioning stylet 586 of the tube assembly guidance system 588, such as the CORTRAK® enteral access system, may be preloaded within the multiple lumen tube 108 such that the second end 598 of the positioning stylet 586 is located at the distal end 344 of the first lumen 320. The preloaded placement of the positioning stylet 586 advantageously allows a clinician to determine the placement of the distal end 314 of the multiple lumen tube 108 within the patient.

As shown in FIGS. 5E and 5F, in another example embodiment, the Y-connector 106 may also incorporate a valve 582 such as a stopcock to selectively control the flow of liquid within the Y-connector 106, or a stopcock type connector may be used to select between channels. Such a connector could be made part of Y-connector 106 with the dual purpose of providing a junction for the administration tubing 150, the suction tubing 120, and the first lumen 320 of the multiple lumen tube 108 and selecting fluid flow in either a path through the suction arm 508 to the suction tubing 120 or through the administration tubing 150 to the delivery arm 504. Using a valve 582, such as a stopcock, may advantageously reduce any chance of improperly using suction tubing 120 or administration tubing 150 connections concurrently because the clinician will have to actively select the fluid flow path. For example, when the valve 582 is positioned in a “delivery arrangement,” as illustrated in FIG. 5E, fluid may flow through the delivery arm 504 to the convergence arm 510, while the flow path to the suction arm 508 is blocked. Additionally, when the valve 582 is positioned in a “suction arrangement,” as illustrated in FIG. 5F, fluid may flow through convergence arm 510 to the suction arm 508, while the flow path to the delivery arm 504 is blocked.

As shown in FIGS. 6A and 6B, in an example embodiment, the inner core 610 may be made substantially or completely of a first material, and may be fitted with a material over-layer 614 unit made substantially or completely of a second material. Additionally, the Y-connector may be made from one material, in a single mold, or multiple materials from several molds.

The inner core 610 defines the various arms, channels and connectors of the Y-connector 106. The Y-connector 106 may be made of a rigid material and construction may include a suitable copolyester or the like. An amorphous copolyester product typically has suitable appearance, clarity and mold release properties (usable with injection molding, for example). It also provides appropriate toughness, hydrolytic stability, heat resistance, and chemical resistance and has been formulated for medical devices. This material (and others like it that can be used in Y-connector 106 embodiments claimed herein) provides a strong inner core 610 for the Y-connector 106, including a suitable small-bore connector 618 that meets appropriate standards, guidelines and/or other requirements (e.g., ISO 80369-1:2010 covering small-bore connectors for liquids and gases in healthcare applications). Also, because the rigid material in some Y-connector 106 embodiments is clear, a user is able to confirm visually that connections have been made, to confirm visually that administration is occurring, and to determine visually the cleanliness of the Y-connector 106 before, during and/or after use. The inner core 610 may also be made of a translucent or opaque material, including materials that can possess preselected color characteristics. The inner core 610 may be a single, unitary plastic structure or may be constructed of several components that are combined by adhesive or solvent bonding.

The over-layer 614 may be made of a pliable material such as a thermoplastic elastomer in some embodiments. When such an elastomer is used, it can be a medical-compliant over-mold that adheres to various substrates, including the types of rigid material used in the inner core 610. This type of material has a rubber feel and soft touch and is clear or translucent. Additionally, the material may also be used in injection molding fabrication. The over-layer 614 may also be made of an opaque material, including materials that can possess preselected color characteristics. The over-layer 614 provides a good gripping material for individuals (e.g., nurse, clinician, caregiver, patient, etc.) who are handling and manipulating the Y-connector 106 and also provides a resilient enclosure that permits the use of a rigid material for the inner core 610 while protecting the inner core 610 from breakage, damage, slipperiness and other undesirable characteristics. Moreover, the soft, flexible material over-layer 614 allows the Y-connector 106 to be placed in close proximity to a patient's face or other exposed skin more comfortably. The combination of the inner core 610 and the over-layer 614 permits an organic, smooth shape that allows for ergonomic gripping of the Y-connector 106 during use.

The Y-connector 106 includes a delivery arm 504 and one or more suction arms 508 that provide fluid flow (i.e., gas flow and/or liquid flow) through the Y-connector 106. As seen in in FIG. 6A and 6B, the delivery arm 504 includes a generally cylindrical delivery arm stem 604 and a delivery arm channel 634. The suction arm 508 includes a generally cylindrical suction arm stem 608 and a suction arm channel 638. The suction arm stem 608 has a proximal end 690 and a distal end 692. The suction arm channel 638 extends through the suction connector 318. The convergence arm 510 includes a generally cylindrical convergence arm stem 620 and a convergence channel 630 (e.g., connection port, which may also be referred to as a connector port). A suction arm small-bore connector 618 may be positioned at the outer end of the suction arm stem 608. For example, the suction connector 318 may be removable and adapted to screw into the small-bore connector 618. In another example embodiment, the suction connector 318 may be permanently coupled to the suction arm 508. Additional suction and/or delivery arms may be implemented in a similar fashion. The suction arm stem 608 defines an interior suction arm channel 638. In some embodiments, each suction arm stem 608 and its associated small-bore connector 618 are components of an integral inner core 610. In other embodiments, the small-bore connector 618 may be affixed to the suction arm stem 608 using adhesive, solvent bonding, radio-frequency (RF) welding and/or any other suitable means of affixing connector 618 to stem 608.

In an example embodiment, the suction arm channel 638 may include a valve 650, such as a one-way check valve or other backflow prevention means can be integrated into the Y-connector 106 to prevent flow of gastric material during suction to flow back through the suction channel 638 to the convergence arm 510. The valve 650 also advantageously prevents improper materials and/or fluids from being introduced through the suction connector 318 on the suction arm 508. In another example embodiment, the valve 650a may be located within the suction connector 318. The valve 650 may be positioned at any location within the suction arm channel 638 such that it is interposed between the proximal end 690 of the suction arm stem 608 and the outlet end 694 of the hollow stem connector 318. In an example embodiment, the valve 650 may begin to open in a pressure range between 0.1 psi and 1.0 psi (e.g., 5 mm Hg to 50 mm Hg). In another example embodiment, the valve 650 may begin to open around 0.25 psi (e.g., has a crack pressure of 0.25 psi or 13 mm Hg).

The inner bore diameter of the interior channels of the delivery arm 504 and the suction arm 508, respectively, may be different from the inner bore diameter of the convergence arm 510 in some embodiments. The interior channel diameters may be dependent upon the type of connection used between the Y-connector and any tubing segment(s) transmitting fluid to or from the Y-connector. As shown in FIGS. 6A and 6B, in an example embodiment, an interior annular neck 640 in the interior of the inner core 610 may define a linking channel 642. The interior annular neck 640 defining a linking channel 642 can serve as an anchoring point for a tubing segment, such as a multiple lumen tube, or similar attachments to the convergence arm. In an example embodiment, the linking channel 642, the convergence channel 630 (e.g., connection port), and the delivery arm channel 634 may all have the same diameter. In some embodiments, the tubing segments, such as a multiple lumen tube 108, or similar attachments can be solvent bonded to one or more interior surfaces of the inner core 610 (e.g., to the annular abutment of the neck and/or to the interior surface of the interior channel). Additionally, in an example embodiment, the tubing segments or multiple lumen tube 108 may be removably press-fit or frictionally fit into the convergence arm. Any suitable means of affixing the Y-connector and the tubing segments may be utilized.

The bores of the delivery arm 504 and suction arm 508 are dictated by dimensions permitted within a given standard or other specific definition (e.g., ISO 80369-3), which can be defined for reducing misconnection potential. The bore size of the convergence arm 510 may be related to the size of the tubing that is attached to it. In an example embodiment, the smallest inner diameter of the convergence arm is no smaller than the inner diameter of the attached tubing. For example, the connection port of the convergence arm 510 may be adapted to receive a multiple lumen tube ranging from size 4 French to size 22 French, although 14 to 18 French sizes are most utilized for nasogastric suctioning applications.

In some Y-connector embodiments usable with enteral feeding systems, for example, the interior channel of the delivery arm (delivery arm channel 634) and the interior channel of the suction arm (suction arm channel 638) may each have an interior bore of approximately 2.90 mm and the inner bore of the interior channel of the convergence arm (convergence channel 630) may be approximately 2.90 mm. In another embodiment, the convergence channel 630 may be larger or may be flared at the end, enabling the convergence channel 630 (e.g., connection port) to fit over larger tubes. In some embodiments, the delivery and suction channels (634, 638) may be connected to the convergence channel 630 using an even smaller diameter linking channel 642. The linking channel 642 may have an inner bore of approximately 2.67 mm and a length of approximately 1.5 mm in some embodiments.

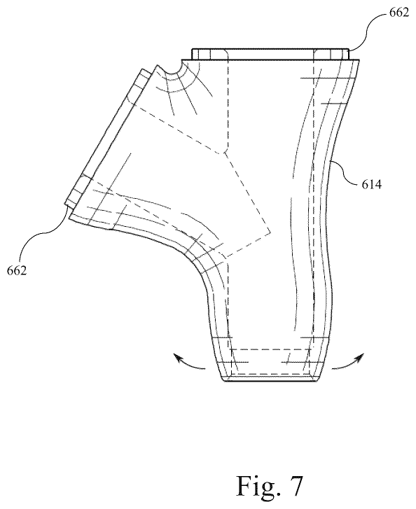

In an example embodiment, a portion of the delivery arm stem 604 may be enclosed in the soft material over-layer 614. As seen in FIG. 6A, FIG. 6B, and FIG. 7, a small circumferential gap 662 is provided between the delivery arm small-bore connector 618 and the portion of the over-layer 614 enclosing the delivery arm stem 604. The gap 662 allows a ring connector 530 or other suitable connector to be attached to the delivery arm 504 so that a small-bore connector cap 540 can be tethered or otherwise attached to the delivery arm 504. The gap 662 enables the ring connector 530 to swing or rotate circumferentially around the small-bore connector 618, thus facilitating movement and positioning of the connector and plug or cap for better access by a clinician or patient to the small-bore connector 618. The gap 662 also ensures that the ring connector 530 remains connected to the Y-connector 106 without the need of a tray, table or other work surface needed to hold the Y-connector ports (e.g., while changing configurations). Additionally, a portion of the suction arm stem 608 is enclosed by an over-layer 614. A small circumferential gap 662 is similarly provided between the suction arm portion of the over-layer and the suction connector 318. The gap 662 allows a ring connector 530 or other suitable connector to be attached to the suction arm 508 so that a suction connector plug 560 or a suction connector cap 562 can be tethered or otherwise attached to the suction arm 508. The gap 662 enables the ring connector to swing or rotate circumferentially around the suction connector 318, thus facilitating movement and positioning of the connector and cap for better access by a clinician or patient to the small-bore connector. The gap 662 also ensures that the ring connector 530 remains with the Y-connector 106.

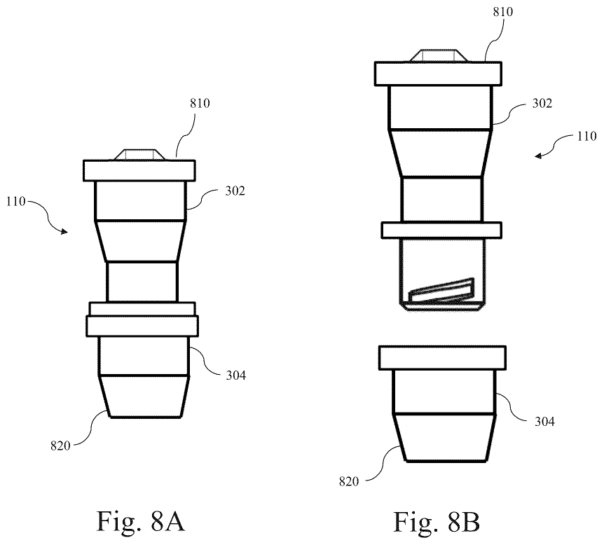

FIG. 8A and FIG. 8B show an anti-reflux valve 110. The anti-reflux valve 110 includes an anti-reflux filter housing 302 and an associated connector 304. The anti-reflux filter housing 302 includes a membrane or vent material that is adapted to allow air to flow into the top end 810 of the anti-reflux valve 110 while preventing fluid from escaping the second lumen 324. For example, the anti-reflux filter housing 302 may include a one-way valve that only allows air and/or a flushing fluid to be introduced into the second lumen 324 from the anti-reflux valve 110. In an example embodiment, the valve may begin to open in a pressure range between 0.1 psi and 1.0 psi (e.g., 5 mm Hg to 50 mm Hg). In another example embodiment, the valve may begin to open around 0.25 psi (e.g., has a crack pressure of 0.25 psi or 13 mm Hg). In another example embodiment, the anti-reflux filter housing may include a membrane or vent material that is adapted to allow air to flow in and out of the top end 810 of the anti-reflux valve 110 to the second lumen 324 and prevent fluid from escaping the second lumen 324. The bottom end 820 of the anti-reflux valve 110 is adapted to couple to the proximal end 350 of the second lumen 324 tube. In an example embodiment, the bottom end 820 of the anti-reflux valve 110 is permanently attached to the multiple lumen tube 108 using an adhesive or any other suitable attachment means. For example, the membrane may include a vent material such as a hydrophobic and/or oliophobic vent materials placed over the sump vent to prevent reflux exit. For example, the vent material may be included in the anti-reflux filter housing 302 as a plug that can be removed from the anti-reflux valve 110 and replaced. Additionally, the anti-reflux valve 110 may include one or more membranes, each membrane may be selected from an appropriate range of materials and configurations (e.g., a hydrophobic and/or oleophobic membrane made of materials such as those produced by Gore and Trinity Technology Group (TTG), for example a membrane material may be an acrylic copolymer membrane cast on a nonwoven nylon support. The membrane material may then be treated for desired performance regarding oleophobicity and/or hydrophobicity.

For example, the breathability (i.e., measure of air permeability—typically measured by the Gurley number), liquid surface tension (force exerted by below-the-surface molecules upon those at the surface-air interface), solids surface-free energy (force exerted by below-the-surface molecules upon those at the surface-air interface), and the membrane's wet-out properties (filling the membrane pores with fluid) are important properties for the membrane. In settings where the membrane is likely to come into frequent contact with fluids such as aspirating fluid or fluid nutrient formula, membrane embodiments are used that resist “wetting” that reduces its efficiency.

In an example embodiment, as illustrated in FIG. 8B, the anti-reflux filter housing 302 may be removed from the anti-reflux valve 110 thereby leaving the connector 304 attached to the second lumen 324 of the multiple lumen tube 108. Once the anti-reflux filter housing 302 is removed, it may be replaced, or other tube connections may be inserted into connector 304, such as connector 250 associated with relief tubing 240 to provide gastric relief or an enteral syringe (e.g., an ISO 80369-3 compliant enteral syringe). The anti-reflux filter housing 302 and the connector 304 may be made of polyamide, polyethylene, polypropylene, polyacrylonitrile, copolyester, or the like, which advantageously provide a rigid structure to help prevent misconnections, provides toughness/durability to the components, have chemical resistant properties, and allow for ease of manufacturability.

The top end 810 of the anti-reflux valve 110 may be adapted to attach to a syringe. For example, the top end 810 of the anti-reflux valve 110 may include a male enteral only connector (e.g., ISO 80369-3) that is adapted to receive an enteral syringe (e.g., an ISO 80369-3 compliant enteral syringe). The syringe may be used to force air through the anti-reflux valve 110 to clean the membrane material in a situation where the filter or membrane material becomes clogged with gastric material after wetting occurs. Additionally, the syringe may be used to force reflux material back through the second lumen before wetting occurs. This advantageously prolongs the usable life the anti-reflux valve 110 and continues to maintain a closed system, reducing the instances of opening the system to contamination when an anti-reflux filter housing 302 needs to be replaced. “Open” systems (e.g. one where the patient's gastric aspirate is open to the external environment) also risk clinician and caregiver exposure to the patient's bodily fluids. Such potential exposure to a patient's bodily fluids also require additional Personal Protective Equipment (PPE) per the Centers for Disease Control and Prevention (CDC) and the National Institute for Occupational Safety and Health (NIOSH).

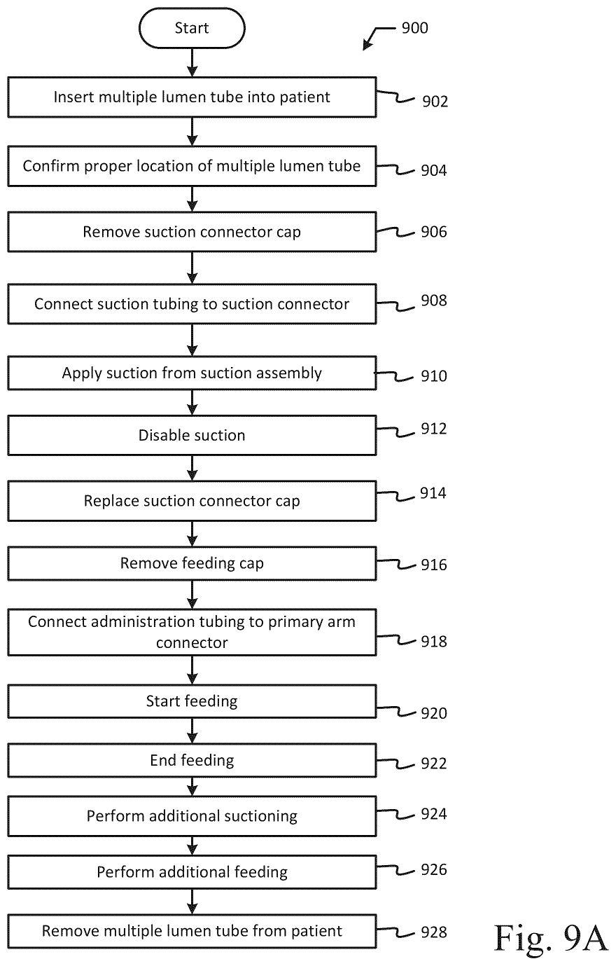

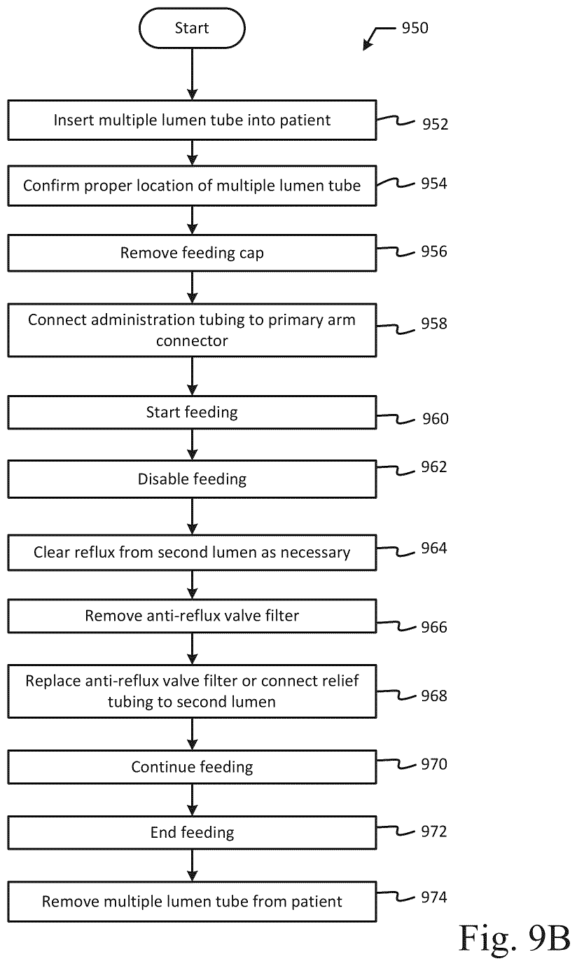

FIG. 9A includes a flowchart of an example suction and feeding process 900 using a nasogastric enteral suction system and feeding system. Although process 900 is described with reference to the flowchart illustrated in FIG. 9A, it will be appreciated that many other methods and sequencing of performing the acts associated with the process 900 may be used. For example, the order of many of the blocks may be changed, many blocks may be intermittently repeated or continually performed, certain blocks may be combined with other blacks, and many of the blocks described are optional or may only be contingently performed.

The example process 900 may begin with a clinician inserting the multiple lumen tube 108 from the gastric tubing system 104 into the patient (block 902). Then, the clinician may confirm the proper location of the multiple lumen tube 108. For example, the clinician may remove the delivery arm connector cap 540 and utilize a positioning stylet 586 of a tube assembly guidance system 588 to confirm the proper location of the multiple lumen tube 108 (block 904). For example, the positioning stylet 586 of the tube assembly guidance system, such as the CORTRAK® enteral access system, may be used with the delivery arm 504 of the gastric tubing apparatus 104 to assist in positioning the closed distal end 314 of the multiple lumen tube 108 within the patient 55. Also, for example, the clinician may use x-ray technology and the radiopaque stripe 380 to confirm placement location. The clinician may also obtain a sample of gastric aspirate using an enteral syringe and check pH levels. Once the position of the closed distal end 314 of the multiple lumen tube 108 is confirmed, the clinician may remove the suction connector plug 560 or suction connector cap 562 (block 906). Then, the clinician may connect the suction tubing 120 to the suction connector 318 (block 908) and apply suction from the suction assembly 112 (block 910). The suction assembly 112 may include a vacuum 116 such as a wall-mounted vacuum that can apply different suction pressures based on the patient and the procedure. For example, a neonatal patient may require and/or tolerate a smaller suction pressure than an adult will. When suction is no longer necessary, the clinician may disable the suction by turning the vacuum 116 off (block 912). Then, the clinician may replace the suction connector cap 562 (block 914). If feeding is necessary, the clinician may remove the feeding cap or the delivery arm connector cap 540 (block 916). Additionally, at this point, the clinician may once again confirm the proper location of the multiple lumen tube 108 by utilizing the positioning stylet 586 or by using radiopaque stripe 380. Then, the clinician may connect the administration tubing 212 to the delivery arm 504 via the small-bore connector 618 (block 918). Then, the clinician may start the feeding procedure (block 920). If gravity is being used to induce feeding flow, the clinician may adjust the appropriate administration tube set clamping mechanism 220 to allow fluid such as nutrient formula to flow through the administration tubing 212. If a feeding pump 216 is being used to induce feeding flow, then the clinician may activate the feeding pump at the required setting. Then, the clinician may end the feeding procedure and turn off the feeding pump 216 or close the administration tube set clamping mechanism 220 (block 922). After feeding has been completed or halted (e.g., due to patient discomfort), it may be necessary or beneficial to perform an additional suction procedure. For example, the clinician may perform the necessary steps from blocks 906 through 912 to perform additional suctioning (block 924). After an additional suction procedure, the clinician may initiate an additional feeding procedure by following any of the necessary steps from blocks 914 through 922 (block 926). Finally, the clinician may remove the multiple lumen tube 108 from the patient (block 928).

FIG. 9B includes a flowchart of example process 950 using an enteral feeding and gastric pressure relief device. Although process 950 is described with reference to the flowchart illustrated in FIG. 9B, it will be appreciated that many other methods of performing the acts associated with the process 950 may be used. For example, the order of many of the blocks may be changed, many blocks may be intermittently repeated or continually performed, certain blocks may be combined with other blacks, and many of the blocks described are optional or may only be contingently performed.

The example process 950 may begin with a clinician inserting the multiple lumen tube 108 from the gastric tubing system 104 into the patient (block 952). Then, the clinician may confirm the proper location of the multiple lumen tube 108. For example, the clinician may remove the delivery arm connector cap 540 and utilize a positioning stylet 586 of a tube assembly guidance system 588 to confirm the proper location of the multiple lumen tube 108 (block 954). For example, the positioning stylet 586 of the tube assembly guidance system 588, such as the CORTRAK® enteral access system, may be used with the delivery arm 504 of the gastric tubing apparatus 104 to assist in positioning the closed distal end 314 of the multiple lumen tube 108 within the patient 55. Also, for example, the clinician may use x-ray technology and the radiopaque stripe 380 to confirm placement location. The clinician may also obtain a sample of gastric aspirate using an enteral syringe and check pH levels. Once the position of the closed distal end 314 of the multiple lumen tube 108 is confirmed, the clinician may remove the feeding cap or the delivery arm connector cap 540 (block 956). Then, the clinician may connect the administration tubing 212 to the delivery arm 504 of the Y-connector 106 (block 958) and start feeding from enteral feeding container 204 (block 960). If the patient is experiencing problems with the feeding, and reflux needs to be relieved, the clinician may disable feeding by closing the appropriate administration tube set clamping mechanism 220 or disabling the feeding pump 216 (block 962). The clinician may clear reflux from the second lumen 324 or vent tube 410 as necessary (block 964). For example, the clinician may attach an enteral syringe containing air to the anti-reflux valve 110 to clear reflux from the second lumen 324 or vent tube 410. Then, the clinician may remove anti-reflux filter housing 302 (block 966). Additionally, the clinician may use a syringe containing air to clear reflux from the second lumen 324 or vent tube 410 and/or to remove any debris on the anti-reflux valve membrane or filter before removing the filter housing 302. After the anti-reflux filter housing 302 has been removed, the associated connector 304 of the anti-reflux valve 110 will still be attached to the air-vent lumen of the multiple lumen tube 108. Then, the clinician may replace the filter housing 302 or connect the connector 250 of the relief tubing 240 into the connector 304 that is associated with the anti-reflux valve 110 and is affixed at the proximal end 350 of the second lumen 324 tube (block 968). Then, the clinician may continue feeding while the refluxed materials are collected in the reflux material collection reservoir 230 (block 970). Next, the clinician may end the feeding procedure when a desired amount of nutrient formula has been delivered to the patient 55 (block 972). Finally, the clinician may remove the multiple lumen tube 108 from the patient (block 974).

It should be understood that various changes and modifications to the example embodiments described herein will be apparent to those skilled in the art. Such changes and modifications can be made without departing from the spirit and scope of the present subject matter and without diminishing its intended advantages. It is therefore intended that such changes and modifications be covered by the appended claims. Also, it should be appreciated that the features of the dependent claims may be embodied in the systems, methods, and apparatus of each of the independent claims.

Aspects of the subject matter described herein may be useful alone or in combination with any one or more of the other aspect described herein. Without limiting the foregoing description, in an exemplary aspect of the present disclosure, a Y-connector for use in a gastric system includes a delivery arm, a suction arm, a one-way valve, and a convergence arm. The suction arm includes a suction arm stem and an outer end. The suction arm stem has a proximal end and a distal end. The suction arm also includes a tapered hollow stem connector adapted to frictionally fit into a section of tubing. The tapered hollow stem connector has a base and an outlet end, and is axially aligned with the suction arm stem. The base of the hollow stem connector is coupled to the outer end of the suction arm, and the tapered hollow stem connector has an outside diameter that decreases from the base of the hollow stem connector to the outlet end of the hollow stem connector. The one-way valve is interposed between the outlet end of the hollow stem connector and the proximal end of the suction arm stem and the valve is adapted to allow fluid to flow in one direction through the suction arm stem. For example, fluid may flow from the base of the hollow stem connector to the outlet end of the hollow stem connector. The delivery arm has a delivery arm stem, and the delivery arm includes a threaded small-bore connector at an outer end of the delivery arm. The convergence arm has a convergence arm stem. Additionally, the convergence arm includes a connection port adapted to receive a multiple lumen tube. The delivery arm stem is in fluid communication with the convergence arm stem, and the suction arm stem is in fluid communication with the convergence arm stem and the delivery arm stem.

In accordance with another exemplary aspect of the present disclosure, which may be used in combination with the preceding aspect, the Y-connector further includes an inner core made of a first material and an over-layer made of a second material. The over-layer encloses at least a portion of the Y-connector body portion.

In accordance with another exemplary aspect of the present disclosure, which may be used in combination with any one or more of the preceding aspects, the over-layer at least partially encloses the inner core.

In accordance with another exemplary aspect of the present disclosure, which may be used in combination with any one or more of the preceding aspects, the first material may be a rigid plastic material.

In accordance with another exemplary aspect of the present disclosure, which may be used in combination with any one or more of the preceding aspects, the second material over-lay may be a pliable material.

In accordance with another exemplary aspect of the present disclosure, which may be used in combination with any one or more of the preceding aspects, the first material may be a transparent copolyester and the second material may be a translucent thermoplastic elastomer.

In accordance with another exemplary aspect of the present disclosure, which may be used in combination with any one or more of the preceding aspects, the outside diameter of the tapered hollow stem connector may decrease in a stepwise fashion.

In accordance with another exemplary aspect of the present disclosure, which may be used in combination with any one or more of the preceding aspects, the outside diameter of the tapered hollow stem connector may decrease in a constant linear fashion.

In accordance with another exemplary aspect of the present disclosure, which may be used in combination with any one or more of the preceding aspects, the connector port of the convergence arm is adapted to receive a multiple lumen tube ranging from size 14 French to size 18 French.

In accordance with another exemplary aspect of the present disclosure, which may be used in combination with any one or more of the preceding aspects, the Y-connector further includes a first cap configured to seal the delivery arm and a second cap configured to seal the tapered hollow stem connector on the suction arm.

In accordance with another exemplary aspect of the present disclosure, which may be used in combination with any one or more of the preceding aspects, the first cap may be coupled to the delivery arm with a first tether configured to circumferentially rotate the first cap about the delivery arm. Additionally, the second cap may be coupled to the suction arm with a second tether configured to circumferentially rotate the second cap about the suction arm.

In accordance with another exemplary aspect of the present disclosure, which may be used in combination with any one or more of the preceding aspects, the second cap is adapted to fit over the entire surface of the tapered hollow stem connector.

In accordance with another exemplary aspect of the present disclosure, which may be used in combination with any one or more of the preceding aspects, the second cap is adapted to fit over an end portion of the tapered hollow stem connector.

In accordance with another exemplary aspect of the present disclosure, which may be used in combination with any one or more of the preceding aspects, the second cap includes a plug adapted to frictionally fit inside the hollow portion of the outer end of the tapered hollow stem connector.

In accordance with another exemplary aspect of the present disclosure, which may be used in combination with any one or more of the preceding aspects, a gastric tubing system includes a Y-connector and a multiple lumen tube. The Y-connector includes a delivery arm, a suction arm, a one-way valve, and a convergence arm. The suction arm includes a suction arm stem and an outer end. The suction arm stem has a proximal end and a distal end. The suction arm also includes a tapered hollow stem connector adapted to frictionally fit into a section of tubing. The tapered hollow stem connector has a base and an outlet end, and is axially aligned with the suction arm stem. The base of the hollow stem connector is coupled to the outer end of the suction arm, and the tapered hollow stem connector has an outside diameter that decreases from the base of the hollow stem connector to the outlet end of the hollow stem connector. The one-way valve is interposed between the outlet end of the hollow stem connector and the proximal end of the suction arm stem, and the valve is adapted to allow fluid to flow in one direction through the suction arm stem. For example, fluid may flow from the base of the hollow stem connector to the outlet end of the hollow stem connector. The delivery arm has a delivery arm stem, and the delivery arm includes a threaded small-bore connector at an outer end of the delivery arm. The convergence arm has a convergence arm stem. Additionally, the convergence arm includes a connection port adapted to receive a multiple lumen tube. The delivery arm stem is in fluid communication with the convergence arm stem, and the suction arm stem is in fluid communication with the convergence arm stem and the delivery arm stem. The multiple lumen tube has an open proximal end and a closed distal end. The open proximal end of the multiple lumen tube is adapted to couple to the connection port on the convergence arm of the Y-connector. Additionally, the multiple lumen tube includes a first lumen and a second lumen that are separated by a wall. The first lumen extends from the proximal end of the multiple lumen tube to the distal end of the multiple lumen tube. The first lumen has an open proximal end and a distal end, the distal end has a plurality of openings. Furthermore, the second lumen has a proximal end and a distal end, the distal end of the second lumen is in fluid communication with the distal end of the first lumen and has a fluid communication opening on the wall between the first lumen and second lumen. The communication opening is adapted to allow air to enter the distal end of the first lumen. The proximal end of the second lumen extends beyond the proximal end of the first lumen, and the proximal end of the second lumen is in fluid communication with the ambient air.

In accordance with another exemplary aspect of the present disclosure, which may be used in combination with the preceding aspect, the proximal end of the second lumen has an enlarged diameter.

In accordance with another exemplary aspect of the present disclosure, which may be used in combination with any one or more of the preceding aspects, the gastric tubing system further includes a positioning stylet. The positioning stylet has a first end and a second end, and also includes a union device. The union device is adapted to couple to the small-bore connector of the delivery arm of the Y-connector. The first end of the positioning stylet includes an electrical connector. The second end of the positioning stylet includes a coil and is adapted to move through the first lumen of the multiple lumen tube. Additionally, the second end of the positioning stylet is located within the first lumen of the multiple lumen tube and is positioned at the distal end of the first lumen.

In accordance with another exemplary aspect of the present disclosure, which may be used in combination with any one or more of the preceding aspects, the gastric tubing system further includes an anti-reflux valve attached to the proximal end of the second lumen.

In accordance with another exemplary aspect of the present disclosure, which may be used in combination with any one or more of the preceding aspects, the anti-reflux valve has a top end and a bottom and includes a one-way valve adapted to allow fluid to flow in one direction through the anti-reflux valve from the top end of the anti-reflux valve to the second lumen.

In accordance with another exemplary aspect of the present disclosure, which may be used in combination with any one or more of the preceding aspects, the anti-reflux valve has a top end and a bottom and includes an anti-reflux housing and a connector. The housing has a membrane that is adapted to obstruct fluid flow while allowing air to flow in both directions. The connector is adapted to receive the anti-reflux housing.

In accordance with another exemplary aspect of the present disclosure, which may be used in combination with any one or more of the preceding aspects, the top end of the anti-reflux valve is adapted to couple to a syringe.

In accordance with another exemplary aspect of the present disclosure, which may be used in combination with any one or more of the preceding aspects, an enteral feeding and suction system includes a fluid source, a suction assembly, a multiple lumen tube, an administration tubing segment and a suction tubing segment. The suction assembly includes a vacuum and a suction canister. Additionally, the vacuum and the suction canister are connected by a section of vacuum tubing. The Y-connector includes a delivery arm, a suction arm, a one-way valve, and a convergence arm. The suction arm includes a suction arm stem and an outer end. The suction arm stem has a proximal end and a distal end. The suction arm also includes a tapered hollow stem connector adapted to frictionally fit into a section of tubing. The tapered hollow stem connector has a base and an outlet end, and is axially aligned with the suction arm stem. The base of the hollow stem connector is coupled to the outer end of the suction arm, and the tapered hollow stem connector has an outside diameter that decreases from the base of the hollow stem connector to the outlet end of the hollow stem connector. The one-way valve is interposed between the outlet end of the hollow stem connector and the proximal end of the suction arm stem and the valve is adapted to allow fluid to flow in one direction through the suction arm stem. For example, fluid may flow from the base of the hollow stem connector to the outlet end of the hollow stem connector. The delivery arm has a delivery arm stem, and the delivery arm includes a threaded small-bore connector at an outer end of the delivery arm. The convergence arm has a convergence arm stem. Additionally, the convergence arm includes a connection port adapted to receive a multiple lumen tube. The delivery arm stem is in fluid communication with the convergence arm stem, and the suction arm stem is in fluid communication with the convergence arm stem and the delivery arm stem. The multiple lumen tube has a proximal end and a distal end. The multiple lumen tube includes a first lumen and a second lumen. The first lumen has a proximal end and a distal end. The distal end has at least one opening. The proximal end of the first lumen is adapted to attach to the connection port on the convergence arm of the Y-connector. The second lumen has a proximal end and a distal end. The distal end of the second lumen is in fluid communication with the distal end of the first lumen and has an opening on the wall between the first lumen and the second lumen adapted to allow air to enter the distal end of the first lumen. The proximal end of the second lumen extends beyond the proximal end of the first lumen, and the proximal end of the second lumen is in fluid communication with the ambient air and has an enlarged diameter. The administration tubing segment has a first end connected to the fluid source and a second end adapted to connect to the primary arm of the Y-connector. The suction tubing segment has a first end and a second end. The first end of the suction tubing segment is connected to the suction canister, and the second end of the suction tubing segment is adapted to connect to the tapered hollow stem connector on the secondary arm.

In accordance with another exemplary aspect of the present disclosure, which may be used in combination with any one or more of the preceding aspect, the enteral feeding and suction system further includes an enteral feeding pump.

In accordance with another exemplary aspect of the present disclosure, which may be used in combination with any one or more of the preceding aspects, the enteral feeding and suction system further includes an anti-reflux valve attached to the proximal end of the second lumen.

In accordance with another exemplary aspect of the present disclosure, which may be used in combination with any one or more of the preceding aspects, an enteral feeding and gastric pressure relief system includes a gastric material collection reservoir, a fluid source, a Y-connector, a multiple lumen tube, an administration tubing segment, and a relief tubing segment. The gastric material collection reservoir is adapted to collect reflux fluids from the patient's stomach. The Y-connector includes a delivery arm, a suction arm, a one-way valve, and a convergence arm. The suction arm includes a suction arm stem and an outer end. The suction arm stem has a proximal end and a distal end. The suction arm also includes a tapered hollow stem connector adapted to frictionally fit into a section of tubing. The tapered hollow stem connector has a base and an outlet end, and is axially aligned with the suction arm stem. The base of the hollow stem connector is coupled to the outer end of the suction arm, and the tapered hollow stem connector has an outside diameter that decreases from the base of the hollow stem connector to the outlet end of the hollow stem connector. The one-way valve is interposed between the outlet end of the hollow stem connector and the proximal end of the suction arm stem and the valve is adapted to allow fluid to flow in one direction through the suction arm stem. For example, fluid may flow from the base of the hollow stem connector to the outlet end of the hollow stem connector. The delivery arm has a delivery arm stem, and the delivery arm includes a threaded small-bore connector at an outer end of the delivery arm. The convergence arm has a convergence arm stem. Additionally, the convergence arm includes a connection port adapted to receive a multiple lumen tube. The delivery arm stem is in fluid communication with the convergence arm stem, and the suction arm stem is in fluid communication with the convergence arm stem and the delivery arm stem. The multiple lumen tube includes a first lumen and a second lumen. The first lumen has a proximal end and a distal end. The distal end has at least one opening. The proximal end of the first lumen is adapted to attach to the connection port on the convergence arm of the Y-connector. The second lumen has a proximal end and a distal end. The distal end of the second lumen is in fluid communication with the distal end of the first lumen and has an opening on the wall between the first lumen and the second lumen adapted to allow air to enter the distal end of the first lumen. The proximal end of the second lumen extends beyond the proximal end of the first lumen, and the proximal end of the second lumen is in fluid communication with the ambient air and has an enlarged diameter. The administration tubing segment has a first end connected to the fluid source and a second end adapted to connect to the delivery arm of the Y-connector. The relief tubing segment has a first end and a second end. The first end of the relief tubing segment is connected to the gastric material collection reservoir. Additionally, the second end of the relief tubing segment is connected to the open proximal end of the second lumen of the multiple lumen tube.

In accordance with another exemplary aspect of the present disclosure, which may be used in combination with the preceding aspect, the system further includes an anti-reflux valve connector attached to the proximal end of the second lumen.

The many features and advantages of the present disclosure are apparent from the written description, and thus, the appended claims are intended to cover all such features and advantages of the disclosure. Further, since numerous modifications and changes will readily occur to those skilled in the art, the present disclosure is not limited to the exact construction and operation as illustrated and described. Therefore, the described embodiments should be taken as illustrative and not restrictive, and the disclosure should not be limited to the details given herein but should be defined by the following claims and their full scope of equivalents, whether foreseeable or unforeseeable now or in the future.

What is claimed is:

1. An enteral feeding and suction system comprising:
a Y-connector including:
a delivery arm defining a delivery arm channel having a first male ISO 80369-3 compatible connector at a distal end thereof;
a suction arm defining a suction arm channel having a second male ISO 80369-3 compatible connector at a distal end thereof; and
a convergence arm defining a convergence channel in fluid communication with both the delivery arm channel and the suction arm channel;
a tapered hollow stem connector comprising a female ISO 80369-3 compatible connector at a first end and mated with the second male ISO 80369-3 compatible connector, wherein the tapered hollow stem connector is axially aligned with the suction arm, and an outlet end of the tapered hollow stem connector is in fluid communication with the convergence arm channel and the delivery arm channel; and
a multiple lumen tube spanning between a proximal end and a closed distal end and comprising a first lumen and a second lumen, wherein the proximal end is received by the convergence arm, and the first lumen defines a plurality of openings in a sidewall at the distal end.

2. The enteral feeding and suction system of claim 1, wherein the plurality of openings include a first pattern of holes defined on a first side of the first lumen, and a second pattern of holes defined on a second side of the first lumen opposite the first side.

3. The enteral feeding and suction system of claim 2, wherein the first pattern of holes and the second pattern of holes are axially aligned.

4. The enteral feeding and suction system of claim 2, wherein the first pattern of holes and the second pattern of holes are axially spaced between a location hole and a fluid communication opening.

5. The enteral feeding and suction system of claim 1, wherein the multiple lumen tube comprises a radiopaque stripe extending linearly between the plurality of openings at the distal end.

6. The enteral feeding and suction system of claim 5, wherein the first lumen comprises a location hole that interrupts the radiopaque stripe at the distal end.

7. The enteral feeding and suction system of claim 1, wherein the first lumen and the second lumen are fluidly separated by a wall, and wherein wall comprises a distalmost fluid communication opening.

8. The enteral feeding and suction system of claim 1, wherein the Y-connector further comprises a one-way valve interposed between the outlet end of the hollow stem connector and the proximal end of the suction arm stem adapted to allow fluid to flow in one direction through the suction arm stem from a base of the hollow stem connector to the outlet end of the hollow stem connector.

9. The enteral feeding and suction system of claim 8, wherein the one-way valve has a crack pressure that ranges from 0.1 psi to 1.0 psi.

10. The enteral feeding and suction system of claim 1, wherein the Y-connector further comprises a stopcock valve configured to fluidly couple the delivery channel and the convergence channel in a first position, and fluidly couple the suction channel and the convergence channel in a second position.

11. The enteral feeding and suction system of claim 1, wherein the Y-connector includes an inner core made of a first material and an over-layer made of a second material, the over-layer enclosing a body portion of the inner core and defining a first annular gap between the first male ISO 80369-3 compatible connector, and a second annular gap between the second male ISO 80369-3 compatible connector.

12. The enteral feeding and suction system of claim 11, wherein the first material is a transparent copolyester and further wherein the second material is a translucent thermoplastic elastomer.

13. The enteral feeding and suction system of claim 11, wherein a first cap is coupled to the delivery arm with a first tether comprising a ring connector configured to circumferentially rotate around the delivery arm within the first gap, and a second cap is coupled to the suction arm with a second tether comprising a ring connector configured to circumferentially rotate about the suction arm within the second gap.

14. The enteral feeding and suction system of claim 1, further comprising a positioning stylet having a first end and a second end, wherein
the positioning stylet includes a union device adapted to couple to the first male ISO 80369-3 compatible connector of the delivery arm,
the first end of the positioning stylet includes an electrical connector,
the second end of the positioning stylet includes a coil that is adapted to move through the first lumen of the multiple lumen tube, and
the second end of the positioning stylet is located within the first lumen of the multiple lumen tube at the distal end of the first lumen.

15. An enteral feeding and suction system comprising:
a fluid source;
a suction assembly having a vacuum and a suction canister, wherein the vacuum and the suction canister are connected by a section of vacuum tubing;
a Y-connector including:
a suction arm having a suction arm stem and an outer end, wherein
the suction arm stem has a proximal end and a distal end,
the suction arm includes a tapered hollow stem connector adapted to frictionally fit into a suction tube connected to the suction assembly,
the tapered hollow stem connector has a base and an outlet end,
the tapered hollow stem connector is axially aligned with the suction arm stem and the base of the tapered hollow stem connector is coupled to the outer end of the suction arm, and
the tapered hollow stem connector has an outside diameter that decreases from the base of the hollow stem connector to the outlet end of the tapered hollow stem connector;
a one-way valve interposed between the outlet end of the tapered hollow stem connector and the proximal end of the suction arm stem adapted to allow fluid to flow in one direction through the suction arm stem from the base of the tapered hollow stem connector to the outlet end of the tapered hollow stem connector;
a delivery arm having a delivery arm stem, wherein the delivery arm includes a threaded small-bore connector at an outer end of the delivery arm;
a convergence arm having a convergence arm stem, wherein
the convergence arm includes a connection port adapted to receive a multiple lumen tube,
the delivery arm stem is in fluid communication with the convergence arm stem, and
the suction arm stem is in fluid communication with the convergence arm stem and the delivery arm stem;
a multiple lumen tube having a proximal end and a distal end, wherein
the multiple lumen tube includes a first lumen and a second lumen,
the first lumen has a proximal end and a distal end, the distal end has at least one opening,
the proximal end of the first lumen adapted to attach to the connection port on the convergence arm of the Y-connector,
the second lumen having a proximal end and a distal end, the distal end of the second lumen in fluid communication with the distal end of the first lumen and having an opening on a wall between the first lumen and second lumen adapted to allow air to enter the distal end of the first lumen,
the proximal end of the second lumen extending beyond the proximal end of the first lumen, and
the proximal end of the second lumen open to ambient air and having an enlarged diameter;
an administration tubing segment, wherein the administration tubing segment has a first end connected to the fluid source and a second end adapted to connect to the delivery arm of the Y-connector; and
a suction tubing segment, wherein
the suction tubing segment has a first end and a second end,
the first end of the suction tubing segment is connected to the suction canister, and
the second end of the suction tubing segment is adapted to connect to the tapered hollow stem connector on the suction arm.

16. The system of claim 15, further comprising an enteral feeding pump.

17. The system of claim 15, further comprising an anti-reflux valve attached to the proximal end of the second lumen, wherein the anti-reflux valve has a top end and a bottom end, the anti-reflux valve including a one-way valve adapted to allow fluid to flow in one direction through the anti-reflux valve from the top end of the anti-reflux valve to the second lumen.

18. The system of claim 15, further comprising an anti-reflux valve attached to the proximal end of the second lumen, wherein the anti-reflux valve has a top end and a bottom end, the anti-reflux valve including:
an anti-reflux filter housing having a membrane; and
a connector, wherein
the connector is adapted to receive the anti-reflux filter housing, and
the membrane is adapted to obstruct fluid flow while allowing air to flow in both directions.

19. An enteral feeding and gastric pressure relief system comprising:
a gastric material collection reservoir, wherein the gastric material collection reservoir is adapted to collect reflux fluids from a patient's stomach;
a fluid source;
a Y-connector including:
a suction arm having a suction arm stem and an outer end, wherein
the suction arm includes a tapered hollow stem connector adapted to frictionally fit into a section of tubing,
the tapered hollow stem connector has a base and an outlet end,
the tapered hollow stem connector is axially aligned with the suction arm stem and the base of the tapered hollow stem connector is coupled to the outer end of the suction arm, and
the tapered hollow stem connector has an outside diameter that decreases from the base of the tapered hollow stem connector to the outlet end of the tapered hollow stem connector;
a one-way valve interposed between the outlet end of the hollow stem connector and the proximal end of the suction arm stem adapted to allow fluid to flow in one direction through the suction arm stem from the base of the tapered hollow stem connector to the outlet end of the tapered hollow stem connector;
a delivery arm having a delivery arm stem, wherein the delivery arm includes a threaded small-bore connector at an outer end of the delivery arm;
a convergence arm having a convergence arm stem, wherein
the convergence arm includes a connection port adapted to receive a multiple lumen tube,
the delivery arm stem is in fluid communication with the convergence arm stem, and
the suction arm stem is in fluid communication with the convergence arm stem and the delivery arm stem;
a multiple lumen tube having a proximal end and a distal end, wherein
the multiple lumen tube includes a first lumen and a second lumen,
the first lumen has a proximal end and a distal end, the distal end has at least one opening,
the proximal end of the first lumen adapted to attach to the connection port on the convergence arm of the Y-connector,
the second lumen having a proximal end and a distal end, the distal end of the second lumen in fluid communication with the distal end of the first lumen and having an opening on a wall between the first lumen and second lumen adapted to allow air to enter the distal end of the first lumen,
the proximal end of the second lumen extending beyond the proximal end of the first lumen, and
the proximal end of the second lumen open to ambient air and having an enlarged diameter;
an administration tubing segment, wherein the administration tubing segment has a first end connected to the fluid source and a second end adapted to connect to the suction arm of the Y-connector; and
a relief tubing segment, wherein
the relief tubing segment has a first end and a second end, and
the first end of the relief tubing segment is connected to the gastric material collection reservoir, and the second end of the relief tubing segment is connected to the open proximal end of the second lumen of the multiple lumen tube.

20. The system of claim 19, further comprising an anti-reflux valve connector attached to the proximal end of the second lumen.
